(19) United States
(12) Patent Application Publication
(10) Pub. No.: US 2025/0134548 A1
(43) Pub. Date: May 1, 2025

(54) ATHERECTOMY BURR ADAPTED FOR RETROGRADE ABLATION WITH LIMITED DRIVESHAFT WINDUP

(71) Applicant: Boston Scientific Scimed, Inc., Maple Grove, MN (US)

(72) Inventors: Daniel Frank Massimini, Brooklyn Park, MN (US); Holden Tranquillo, Arden Hills, MN (US); Scott Hansen, Maple Grove, MN (US); Andrew David Bicek, Elk River, MN (US)

(73) Assignee: Boston Scientific Scimed, Inc., Maple Grove, MN (US)

(21) Appl. No.: 18/929,822

(22) Filed: Oct. 29, 2024

Related U.S. Application Data
(60) Provisional application No. 63/594,304, filed on Oct. 30, 2023.

(51) Int. Cl. A61B 17/3207; A61B 17/32

(52) U.S. Cl. CPC A61B 17/320758; A61B 2017/320008

(57) ABSTRACT

An atherectomy burr adapted for use in an atherectomy system includes an atherectomy burr body adapted to be secured to a driveshaft. The atherectomy burr body has an outer surface defining a distal tapered surface and a proximal tapered surface, where the distal tapered surface has a first abrasiveness adapted for anterograde ablation and the proximal tapered surface has a second abrasiveness adapted for retrograde ablation. In some cases, the first abrasiveness is greater than the second abrasiveness.

### CROSS-REFERENCE TO RELATED APPLICATIONS

This application claims the benefit of priority under 35 U.S.C. § 119 of U.S. Provisional Application No. 63/594,304, filed Oct. 30, 2023, the entire disclosure of which is hereby incorporated by reference.

### TECHNICAL FIELD

The present disclosure pertains to medical devices, and methods for manufacturing and using medical devices. More particularly, the present disclosure pertains to rotational medical devices, methods, and systems.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an atherectomy burr adapted for use in an atherectomy system. The atherectomy burr includes an atherectomy burr body that is adapted to be secured to a driveshaft. The atherectomy burr body has an outer surface defining a distal tapered surface and a proximal tapered surface. The distal tapered surface has a first abrasiveness adapted for anterograde ablation and the proximal tapered surface has a second abrasiveness adapted for retrograde ablation.

Alternatively or additionally, the first abrasiveness may be greater than the second abrasiveness.

Alternatively or additionally, the distal tapered surface may include abrasive particles having a first average particle size and the proximal tapered surface may include abrasive particles having a second average particle size smaller than the first average particle size.

Alternatively or additionally, the distal tapered surface may include abrasive particles having a first average exposed particle height and the proximal tapered surface may include abrasive particles having a second average exposed particle height that is less than the first average exposed particle height.

Alternatively or additionally, the distal surface may include abrasive particles having an average diameter embedded in a first thickness of overcoat and the proximal surface may include abrasive particles having the same average diameter embedded in a second thickness of overcoat that is thicker than the first thickness of overcoat.

Alternatively or additionally, the distal surface may include abrasive particles having a first average diameter embedded in a thickness of overcoat and the proximal surface may include abrasive particles having a second average diameter embedded in the same thickness of overcoat, where the second average diameter is less than the first average diameter.

Alternatively or additionally, the distal tapered surface may include abrasive particles having a first particle density value and the proximal tapered surface may include abrasive particles having a second particle density value that is less than the first particle density value.

Alternatively or additionally, the distal tapered surface may include abrasive particles laid out in a constant pattern and the proximal tapered surface may include abrasive particles laid out in a discontinuous pattern.

Another example may be found in an atherectomy system. The atherectomy system includes an advancer assembly, a drive assembly adapted to translate relative to the advancer assembly, a knob extending from the drive assembly such that translating the knob results in the drive assembly translating relative to the advancer assembly, and a driveshaft operably coupled with the drive assembly, the driveshaft translating relative to the advancer assembly as the drive assembly translates relative to the advancer assembly. The atherectomy system includes an atherectomy burr.

Alternatively or additionally, the atherectomy burr body may define an axially extending void into which the driveshaft is adapted to be secured.

Another example may be found in an atherectomy system. The atherectomy system includes an advancer assembly, a drive assembly adapted to translate relative to the advancer assembly, a knob extending from the drive assembly such that translating the knob results in the drive assembly translating relative to the advancer assembly, and a driveshaft operably coupled with the drive assembly, the driveshaft translating relative to the advancer assembly as the drive assembly translates relative to the advancer assembly. An atherectomy burr is operably coupled with the driveshaft and is adapted to limit driveshaft windup during retrograde ablation.

Alternatively or additionally, the atherectomy burr may include a distal tapered surface adapted for anterograde ablation and a proximal tapered surface adapted for retrograde ablation.

Alternatively or additionally, the distal tapered surface may have a first abrasiveness and the proximal tapered surface may have a second abrasiveness that is less than the first abrasiveness.

Alternatively or additionally, the distal tapered surface may include abrasive particles having a first average particle size and the proximal tapered surface may include abrasive particles having a second average particle size smaller than the first average particle size.

Alternatively or additionally, the distal tapered surface may include abrasive particles having a first average exposed particle height and the proximal tapered surface may include abrasive particles having a second average exposed particle height that is less than the first average exposed particle height.

Alternatively or additionally, the distal tapered surface may include abrasive particles having a first particle density value and the proximal tapered surface may include abrasive particles having a second particle density value that is less than the first particle density value.

Alternatively or additionally, the distal tapered surface may include abrasive particles laid out in a constant pattern and the proximal tapered surface may include abrasive particles laid out in a discontinuous pattern.

Another example may be found in an atherectomy system. The atherectomy system includes an atherectomy burr including a distal outer surface having a first abrasiveness adapted for anterograde ablation and a proximal outer surface having a second abrasiveness adapted for retrograde ablation. The atherectomy system includes a drive mechanism adapted to rotatably actuate the atherectomy burr in an anterograde ablation direction and/or in a retrograde ablation direction.

Alternatively or additionally, the first abrasiveness may be greater than the second abrasiveness.

Alternatively or additionally, the drive mechanism may include a drive coil adapted to be coupled with the atherectomy burr and a drive assembly adapted to rotatably actuate the drive coil.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic diagram showing an illustrative atherectomy system;

FIG. 2 is a schematic diagram showing features of the illustrative atherectomy system of FIG. 1;

FIG. 3 is a schematic view of an illustrative atherectomy burr usable as part of the illustrative atherectomy system of FIG. 1;

FIG. 4 is a cross-sectional view taken along the line 4-4 of FIG. 3;

FIGS. 5A through 5C are schematic cross-sectional views taken along the line 5-5 of FIG. 3; and

FIG. 6 is a schematic view of an illustrative atherectomy burr usable as part of the illustrative atherectomy system of FIG. 1.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Cardiovascular disease and peripheral arterial disease may arise from accumulation of atheromatous material on the inner walls of vascular lumens, resulting in a condition known as atherosclerosis. Atheromatous and other vascular deposits may restrict blood flow and can cause ischemia in a heart of a patient, vasculature of a patient's legs, a patient's carotid artery, etc. Such ischemia may lead to pain, swelling, wounds that will not heal, amputation, stroke, myocardial infarction, and/or other conditions.

Atheromatous deposits may have widely varying properties, with some deposits being relatively soft and others being fibrous and/or calcified. In the latter case, the deposits may be referred to as plaque. Atherosclerosis occurs naturally as a result of aging, but may also be aggravated by factors such as diet, hypertension, heredity, vascular injury, and the like. Atherosclerosis may be treated in a variety of ways, including drugs, bypass surgery, and/or a variety of catheter-based approaches that may rely on intravascular widening or removal of the atheromatous or other material occluding the blood vessel. Atherectomy is a catheter-based intervention that may be used to treat atherosclerosis.

Atherectomy is an interventional medical procedure performed to restore a flow of blood through a portion of a patient's vasculature that has been blocked by plaque or other material (e.g., blocked by an occlusion). In an atherectomy procedure, a device on an end of a drive shaft that is used to engage and/or remove (e.g., abrade, grind, cut, shave, etc.) plaque or other material from a patient's vessel (e.g., artery or vein). In some cases, the device on an end of the drive shaft may be abrasive and/or may otherwise be configured to remove plaque from a vessel wall or other obstruction in a vessel when the device is rotating and engages the plaque or other obstruction. In some cases, atherectomy involves using an abrasive atherectomy burr that is rotated at high speeds exceeding 100,000 revolutions per minute (RPM) in order to abrade plaque and other hardened materials from within the patient's vessel. Atherectomy burrs may be rotated at speeds exceeding 140,000 RPM, at speeds exceeding 180,000 RPM and even at speeds as high as 220,000 RPM. Atherectomy may include orbital atherectomy in addition to rotational atherectomy.

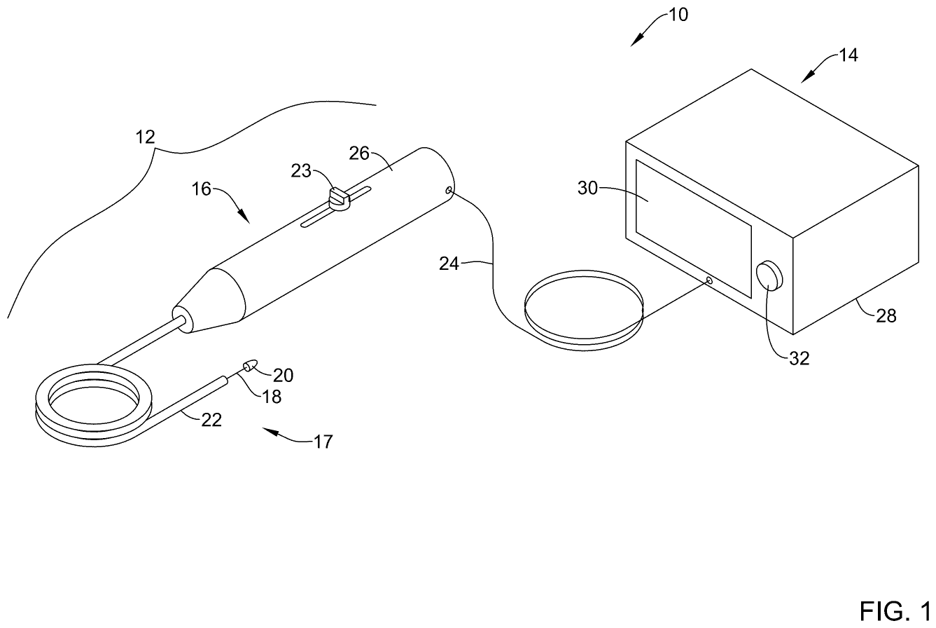

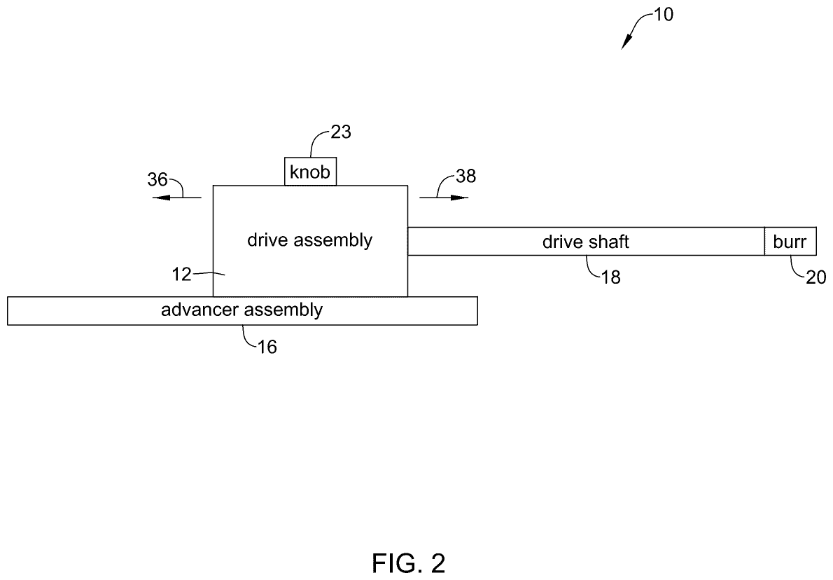

FIGS. 1 and 2 depict an atherectomy system 10. The atherectomy system 10 may be electrically driven, pneumatically driven and/or driven in one or more other suitable manners. Additional or alternative components to those illustrated and described herein may be utilized in the operation of the atherectomy system 10. The atherectomy system 10 may include a drive assembly 12 and a control unit 14 (e.g., a controller). The drive assembly 12 may include, among other elements, an advancer assembly 16 and a rotation assembly 17. Although the control unit 14 is depicted as being separate from the drive assembly 12 in FIG. 1, the functionality of the control unit 14 and the drive assembly 12 may be incorporated into a single component (e.g., in the advancer assembly 16 or other suitable single component).

The rotation assembly 17 may include a drive shaft 18 (e.g., an elongate member that may be or may include a flexible driveshaft or other suitable driveshaft), an atherectomy burr 20 and an elongate member 22 having a first end (e.g., a proximal end), a second end (e.g., a distal end), and a lumen extending from the first end to the second end for receiving the drive shaft 18. In some cases, the elongate member 22 may be an elongated tubular member. The atherectomy burr 20 may have a rough or sharp surface, such that it is configured to grind, abrade, cut, shave, etc. plaque from a vessel wall or other obstruction in a vessel when it is rotated.

The advancer assembly 16 may include a knob 23, a housing 26, the drive assembly 12 and/or one or more other suitable components. In some instances, the drive assembly 12 may be or may include a motor (e.g., an electric motor, pneumatic motor, or other suitable motor) at least partially housed within the housing 26 and in communication with the knob 23, the drive shaft 18, and the control unit 14. In some cases, the motive force may not be disposed within the drive assembly 12, but may instead be remotely located, with a flexible drive cable extending from the motive force to the drive assembly 12. The knob 23 may be configured to advance along a longitudinal path to longitudinally advance the drive assembly 12 and the rotation assembly 17. The housing 26 may at least partially house the drive assembly 12 and the knob 23 may be at least partially accessible from an exterior of the housing 26.

In some instances, the drive assembly 12 is adapted to be translationally secured relative to an advancer assembly 16. In some instances, the advancer assembly 16 may be adapted to be fixed in space, such as being secured to a table, for example. In some instances, the advancer assembly 16 may be part of an advancer housing such as the housing 26. The drive assembly 12 may also be disposed within an advancer handle, for example, but is adapted to translate back and forth (left and right in the illustrated orientation) as indicated by arrows 36 and 38 in response to a user moving the knob 23 in the directions indicated by the arrows 36 and 38. In some instances, as the drive assembly 12 moves back and forth, the drive shaft 18 also moves correspondingly.

The drive assembly 12 may be coupled to the drive shaft 18 in a suitable manner including, but not limited to, a weld connection, a clamping connection, an adhesive connection, a threaded connection, and/or other suitable connection configured to withstand rotational speeds and forces. The drive shaft 18 may be formed from one or more of a variety of materials. For example, the drive shaft 18 may be formed from one or more of a variety of materials, including steel, stainless steel, other metal, polymer, and/or other suitable materials. The drive shaft 18 may have a suitable diameter and/or length for traversing vasculature of a patient. The diameter and/or the length of the drive shaft 18 may depend on the dimension of the lumen of the elongate member 22, the dimensions of vessels of a patient to be traversed, and/or one or more other suitable factors. In some cases, the drive shaft 18 may have a diameter in a range from about 0.030 centimeters (cm) or smaller to about 0.150 cm or larger and a working length in a range from about ten (10) cm or shorter to about three hundred (300) cm or longer. In one example, the drive shaft 18 may have a diameter of about 0.07493 cm and a length of about one hundred forty seven (147) cm. Alternatively, the drive shaft 18 may have a different suitable diameter and/or different suitable length.

The atherectomy burr 20 may have an outer perimeter which is equal to or larger than a distal diameter of the drive shaft 18 and/or the elongate member 22. Alternatively or in addition, the atherectomy burr 20 may have an outer perimeter which is smaller than a diameter of the drive shaft 18 and/or the elongate member 22. The atherectomy burr 20 may be coupled to the drive shaft 18. Where the drive shaft 18 has a first end portion (e.g., a proximal end portion) and a second end portion (e.g., a distal end portion), the atherectomy burr 20 may be coupled to the drive shaft 18 at or near the second end portion. In some cases, the atherectomy burr 20 may be located at or adjacent a terminal end of the second end portion of the drive shaft 18. The atherectomy burr 20 may be coupled to the drive shaft 18 in any manner. For example, the atherectomy burr 20 may be coupled to the drive shaft 18 with an adhesive connection, a threaded connection, a weld connection, a clamping connection, and/or other suitable connection configured to withstand rotational speeds and forces. Similar to as discussed above with respect to the connection between the drive shaft 18 and the drive mechanism, as the drive shaft 18 and/or the atherectomy burr 20 may rotate at speeds between zero (0) RPM and 250,000 RPM or higher, the coupling between the drive shaft 18 and the atherectomy burr 20 may be configured to withstand such rotational speeds and associated forces.

The drive assembly 12 and the control unit 14 may be in communication and may be located in or may have a same housing and/or located in or have separate housings (e.g., the advancer assembly housing 26 and a control unit housing 28 or other housings). Whether in the same housing or in separate housings, the drive assembly 12 and the control unit 14 may be in communication through a wired connection (e.g., via one or more wires in an electrical connector 24 or other suitable electrical connector) and/or a wireless connection. Wireless connections may be made via one or more communication protocols including, but not limited to, cellular communication, ZigBec, Bluetooth, Wi-Fi, Infrared Data Association (IrDA), dedicated short range communication (DSRC), EnOcean, and/or any other suitable common or proprietary wireless protocol, as desired.

Although not necessarily shown in FIG. 1, the drive assembly 12 may include and/or enclose one or more operational features. For example, among other features, the drive assembly 12 may include a motor (e.g., as discussed above and/or other suitable motor), rubber feet, control electronics, drive circuitry, etc.

The control unit 14, which may be separate from the drive assembly 12 (e.g., as shown in FIG. 1) or may be included in the drive assembly 12, may include several features. For example, as shown in FIG. 1, the control unit 14 may include a display 30 and a control knob 32 (e.g., a motor speed (e.g., RPM or other speed) adjustment knob or other control knob). Additionally or alternatively, the control unit 14 may include one or more other features for controlling the drive mechanism and/or other features of the drive assembly 12 (e.g., one or more drive mechanism states of the drive mechanism) including, but not limited to, a processor, memory, input/output devices, a speaker, volume control buttons, on/off power supply switch, motor activation switch, a timer, a clock, and/or one or more other features.

In some cases, the control unit 14 may include one or more drive mechanism load output control mechanisms for controlling an operation of the atherectomy system 10. In one example of a drive mechanism load output control mechanism that may be included in the control unit 14, the control unit 14 may include a mechanism configured to set and/or adjust an advancing load output (e.g., a rotational speed) and/or a retracting load output from the drive assembly 12. Additionally or alternatively, the control unit 14 may include other control and/or safety mechanism for controlling the operation of the atherectomy system 10 and mitigating risks to patients.

In some instances, the drive shaft 18 may be a coil spring. As such, it will be appreciated that rotation of the drive shaft 18 in a first direction may result in the drive shaft 18 undergoing compression, particularly when the first direction corresponds to tightening the individual windings of the coil spring. Rotation of the drive shaft 18 in a second, opposing, direction may result in the drive shaft 18 undergoing tension, particularly when the second direction corresponds to loosening the individual windings of the coil spring. Direction of rotation may come into play particularly when changing from anterograde ablation (in a forward direction) to retrograde ablation (in a reverse direction). An operator may switch from anterograde ablation to retrograde ablation in situations in which the atherectomy burr 20 has popped through a lesion and is located distal of the lesion. Switching to retrograde ablation may assist in being able to move the atherectomy burr 20 proximally back to the proximal side of the lesion. However, because of the differences in how the drive shaft 18 reacts to the forces applied during retrograde ablation, as opposed to anterograde ablation, means that a forward portion of the atherectomy burr 20 may be adapted to have a first level of abrasiveness while the rearward portion of the atherectomy burr 20 may be adapted to have a second level of abrasiveness that is less than the first level of abrasiveness. This can be one way to prevent retrograde ablation from applying too much torque to the drive shaft 18. Subsequent drawings will provide examples of providing the atherectomy burr 20 with varying levels of abrasiveness.

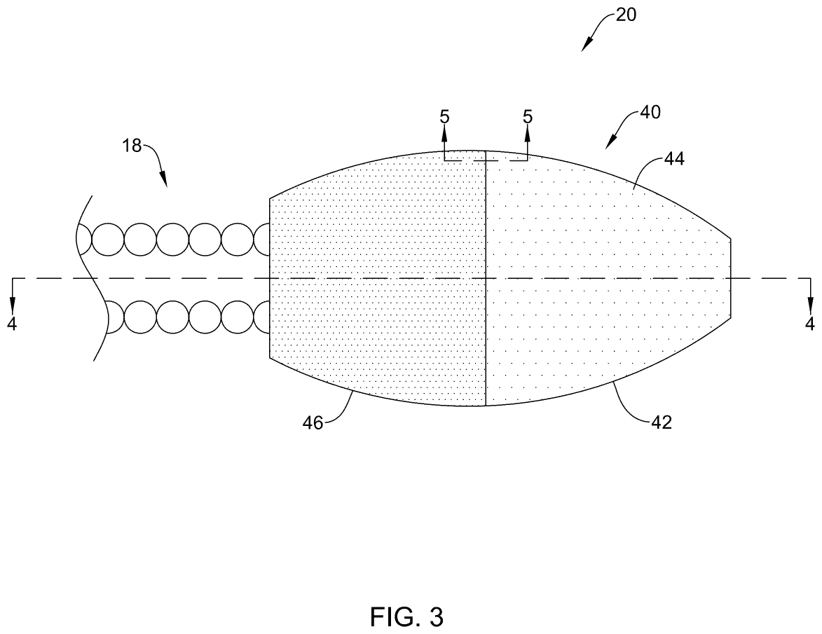

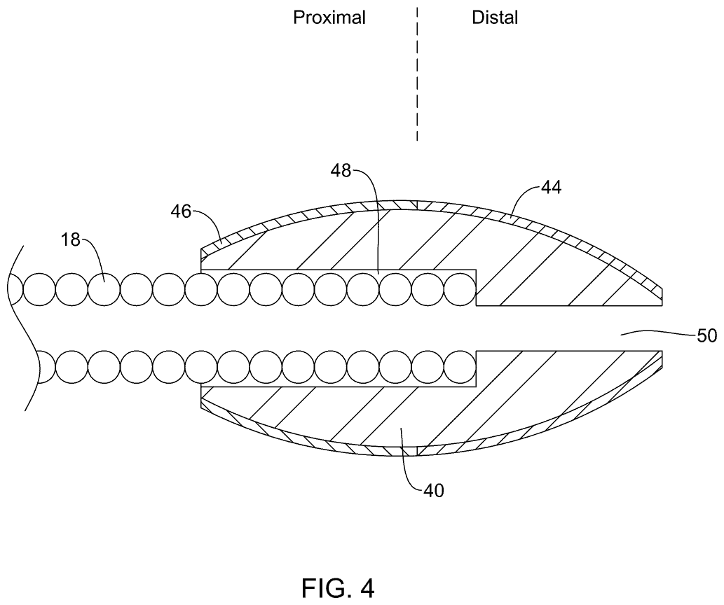

FIG. 3 is a schematic view of the atherectomy burr 20, shown coupled with the drive shaft 18. FIG. 4 is a cross-sectional view through the atherectomy burr 20, taken along the line 4-4 of FIG. 3. In this example, the drive shaft 18 is a coil spring. The atherectomy burr 20 includes an atherectomy body 40 having an outer surface 42. The outer surface 42 may be considered as being divided into a distal tapered surface 44 and a proximal tapered surface 46. In some instances, the atherectomy body 40 defines a void 48 that is adapted to accommodate the drive shaft 18 and to secure the atherectomy burr 20 relative to the drive shaft 18. The atherectomy burr 20 may be adhesively secured, for example, or may be welded or soldered into place. The atherectomy burr body 40 also defines a guidewire lumen 50 that is axially aligned with the void 48 such that a guidewire (not shown) extending within the drive shaft 18 is able to extend through the atherectomy burr body 40.

In some instances, the distal tapered surface 44 may be adapted to be abrasive for performing anterograde ablation when the atherectomy burr 20 is moving in a forward or distal direction and the proximal tapered surface 46 may be adapted to be abrasive for performing retrograde ablation when the atherectomy burr 20 is moving in a backward or proximal direction. During anterograde ablation, the proximal tapered surface 46 will likely not be contacting tissue or the lesion. During retrograde ablation, the distal tapered surface 44 will likely not be contacting tissue or the lesion. In some instances, as will be discussed, the distal tapered surface 44 may be adapted to have an abrasiveness level that is greater than a corresponding abrasiveness level of the proximal tapered surface 46.

Abrasiveness or abrasiveness level may be defined in terms of how well a particular surface abrades or removes material from another surface when the two surfaces are brought into contact and one surface is moved relative to the other surface. A surface having abrasive particles of a first size may be considered as being more abrasive than a surface having abrasive particles of a second, smaller size. A surface having a higher density of abrasive particles may be considered as being more abrasive than a surface having a lower density of the same abrasive particles. In some instances, a comparison may be made with sandpaper. A sandpaper having a lower grit size (larger particle size) is more abrasive and removes material more quickly (and removes larger particles of material) than sandpaper having a higher grit size (smaller particle size). As an example, 100 grit sandpaper is more abrasive than 400 grit sandpaper because the 100 grit sandpaper has larger abrasive particles than the 400 grit sandpaper.

In some instances, grit size may be used to quantify abrasiveness. Grit size is an indication of how many particles of a given size will fit through a particular opening. Hence, a larger particle size yields a lower grit size. As an example, the abrasive particles may have an average diameter that is in a range of less than about 1 μm to about 500 μm, or from about 10 μm to about 50 μm, or between about 20 μm to about 30 μm. Because the abrasive particles are held within an overcoat that helps to secure the abrasive particles in place relative to the atherectomy burr 20, only a portion of the abrasive particles are exposed. As an example, the exposed portion of the abrasive particles may have an average exposed height that is in a range of less than about 1 μm to about 90 μm. This corresponds to a grit size ranging from about 14,000 grit to about 220 grit. The exposed portion of the abrasive particles may have an average exposed height that is in a range of about 2 μm to about 15 μm. The exposed portion of the abrasive particles may have an average exposed height that is in a range of about 5 μm to about 10 μm.

Of course, there can be a tradeoff between abrasiveness and performance, particularly for retrograde ablation. A more abrasive surface may be better and faster at removing material, but can exert a greater torque on the surface being abraded. The relative effect of this, whether the drive shaft 18 is placed into compression (during anterograde ablation) or in tension (during retrograde ablation), plays a part when switching from anterograde ablation to retrograde ablation. Applying too much torque to the drive shaft 18 during retrograde ablation 18 may cause excessive windup (unwinding) of the drive shaft 18. Accordingly, the atherectomy burr 20 may be adapted to limit windup during retrograde ablation. In some instances, this may include providing the proximal tapered surface 46 with less abrasiveness relative to that provided to the distal tapered surface 44.

In some instances, the proximal tapered surface 46 may include a first type of abrasive particles and the distal tapered surface 44 may include a second type of abrasive particles. In some instances, the proximal tapered surface 46 may be made to be less abrasive than the distal tapered surface 44 by virtue of using abrasive particles that are made of a different material than those used for the distal tapered surface 44. The abrasive particles used for the proximal tapered surface 46 may be smaller than the abrasive particles used for the distal tapered surface 44. The abrasive particles used for the proximal tapered surface 44 may be the same size as those used for the distal tapered surface 44. In some instances, a first overcoat material may be used for the proximal tapered surface 46 and a second overcoat material may be used for the distal tapered surface 44 in combination with either the same abrasive particles for both the proximal tapered surface 46 and the distal tapered surface 44, or with different abrasive particles.

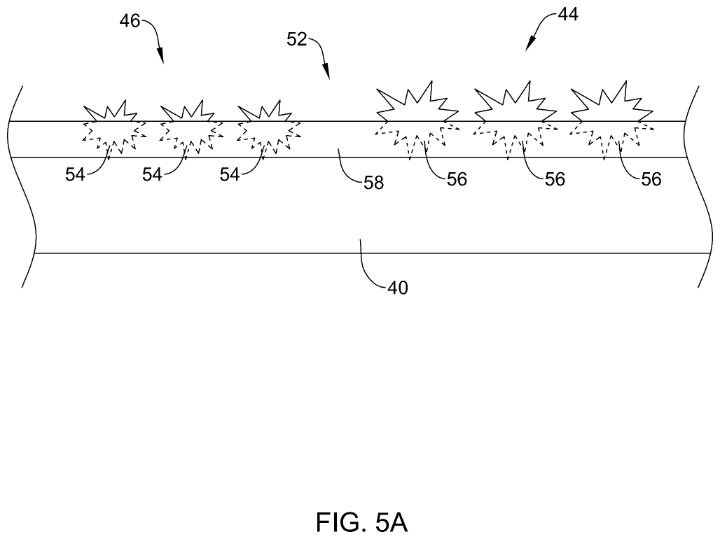

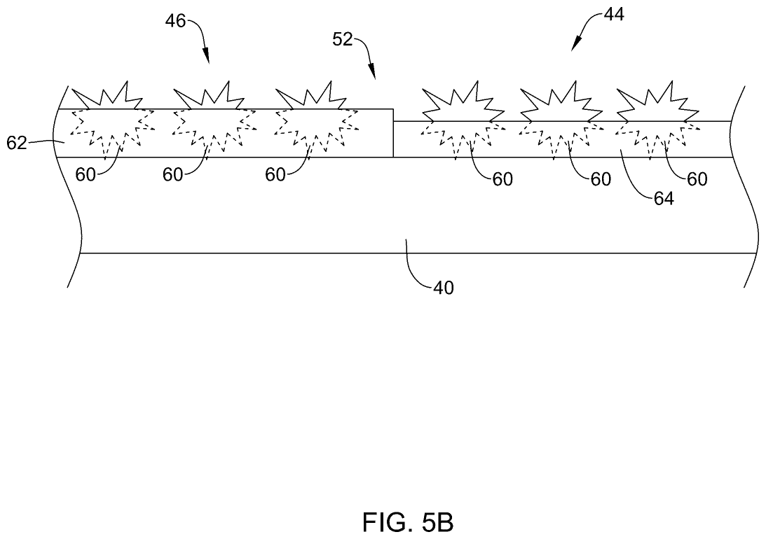

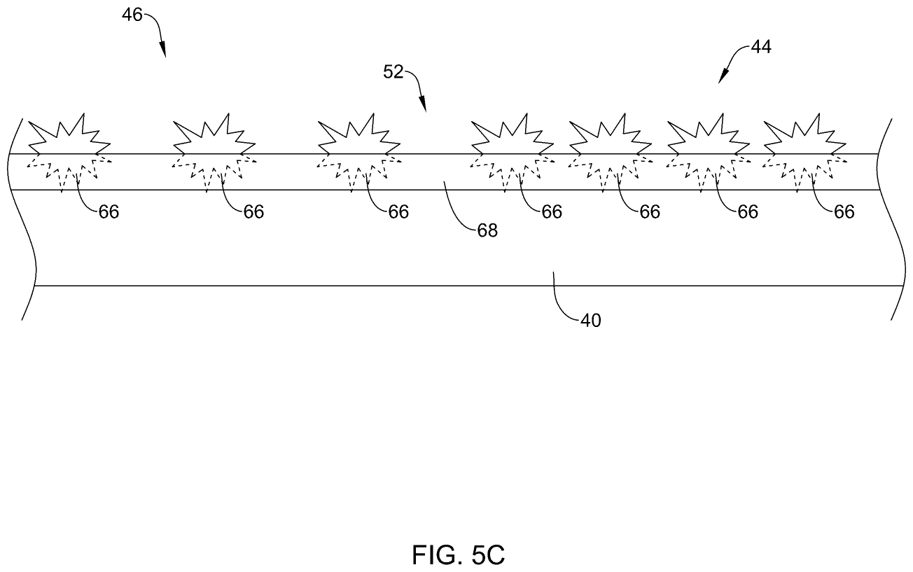

FIG. 5A is a schematic cross-sectional view through a portion of the atherectomy burr 20, providing a close-up view of a transition 52 between the distal tapered surface 44 and the proximal tapered surface 46. A number of abrasive particles 54 are shown within the proximal tapered surface 46. A number of abrasive particles 56 are shown within the distal tapered surface 44. The abrasive particles 54 can be seen as having an overall size that is smaller than the corresponding size of each of the abrasive particles 56. While the abrasive particles 54 are all shown as being the same size, and all of the abrasive particles 56 are all shown as being (a larger) same size, it will be appreciated that there may be a size distribution within the abrasive particles 54, and another size distribution within the abrasive particles 56. The proximal tapered surface 46 may be considered as being less abrasive than the distal tapered surface 44. The abrasive particles 54 and the abrasive particles 56 may be considered as being partially entrapped within an overcoat layer 58. In some instances, the abrasive particles 54 may have an average size in a range of 5 μm to 25 μm, or in a range of 10 μm to 20 μm, and the abrasive particles 56 may have an average size in a range of 15 μm to 40 μm, or in a range of 20 μm to 30 μm. The overcoat layer 58 may be made of nickel, and may have a thickness that is in a range of 10 μm to 25 μm, or in a range of 15 μm to 20 μm. In some instances, the abrasive particles 54 and the abrasive particles 56 may each be diamond particles, for example.

A variety of other abrasive particles may be used, instead of diamond particles or in combination with diamond particles. Examples of other abrasive particles include aluminum oxide, white aluminum oxide, silicone carbide, ceramic alumina, zirconia alumina as well as blends of any of these materials. Other materials may also be used for forming the overcoat layer 58, in addition to nickel. Examples include copper-based alloys, gold-based alloys, palladium-based alloys, inorganic materials, and others. Examples of copper-based alloys include Cu—Sn and Cu—Sn—Zn alloys. Examples of gold-based alloys include Au—Cu—Pd, Au—Cd—Ag, Au—Fe, Au—In, Au—Pd, Au—Ag and Au—Sn alloys. Examples of palladium-based alloys include Pd—Co, Pd—Ni and Pd—Zn alloys. Examples of inorganics include amorphous nickel, Ni—Fe alloys and TiN. These materials may be applied via electrodeposition, physical vapor deposition and powder coating (metallic paint).

FIG. 5B is a schematic cross-sectional view through a portion of the atherectomy burr 20, providing a close-up view of the transition 52 between the distal tapered surface 44 and the proximal tapered surface 46. A number of abrasive particles 60 are shown within the proximal tapered surface 46 and within the distal tapered surface 44. In this example, the abrasive particles 60 are all the same size. This is schematic, as the abrasive particles 60 may have a distribution of average particle sizes, and may not all be exactly the same size. The abrasive particles 60 within the proximal tapered surface 46 are partially embedded in an overcoat layer 62 having a thickness while the abrasive particles 60 within the distal tapered surface 44 are partially embedded in an overcoat layer 64 having a thickness that is less than the thickness of the proximal tapered surface 46. While a different in thickness between the overcoat layer 62 and the overcoat layer 64 is shown in step-wise fashion, this may also be a smooth transition, for example.

Because all of the abrasive particles 60 are roughly the same size, this means that the abrasive particles 60 within the proximal tapered surface 46 have an exposed height that is less than an exposed height of the abrasive particles 60 within the distal tapered surface 44. In some instances, due to size variability, the abrasive particles 60 within the proximal tapered surface 46 may be considered as having an average exposed particle height that is less than an average exposed particle height for the abrasive particles 60 within the distal tapered surface 44. As a result, the proximal tapered surface 46 may be considered as being less abrasive than the distal tapered surface 44. In some instances, the abrasive particles 60 may have an average size in a range of 15 μm to 35 μm, or in a range of 20 μm to 30 μm. The overcoat layer 62 may be made of nickel or other materials, and may have a thickness that is in a range of 20 μm to 35 μm, or in a range of 25 μm to 30 μm. The overcoat layer 64 may be made of nickel or other materials, and may have a thickness that is in a range of 10 μm to 25 μm, or in a range of 15 μm to 20 μm. The abrasive particles 60 may be diamond particles, for example.

FIG. 5C is a schematic cross-sectional view through a portion of the atherectomy burr 20, providing a close-up view of a transition 52 between the distal tapered surface 44 and the proximal tapered surface 46. A number of abrasive particles 66 are shown within the proximal tapered surface 46 and are distributed at a first density (represented by lateral distance between adjacent abrasive particles 66) within the proximal tapered surface 46 and are distributed at a second density (represented by lateral distance between adjacent abrasive particles 66) within the distal tapered surface 44. The first density may be lower than the second density. While each of the abrasive particles 66 are shown as being all the same size, it will be appreciated that there may be a size distribution within the abrasive particles 66. In some instances, the abrasive particles 66 within the proximal tapered surface 46 may be smaller, or be more deeply embedded within an overcoat layer 68, than the abrasive particles 66 within the distal tapered surface 44. As a result, the proximal tapered surface 46 may be considered as being less abrasive than the distal tapered surface 44. In some instances, the abrasive particles 66 may have an average size in a range of 15 μm to 35 μm, or in a range of 20 μm to 30 μm. The overcoat layer 68 may be made of nickel or other materials, and may have a thickness that is in a range of 10 μm to 25 μm, or in a range of 15 μm to 20 μm. In some instances, the abrasive particles 66 may be diamond particles, for example.

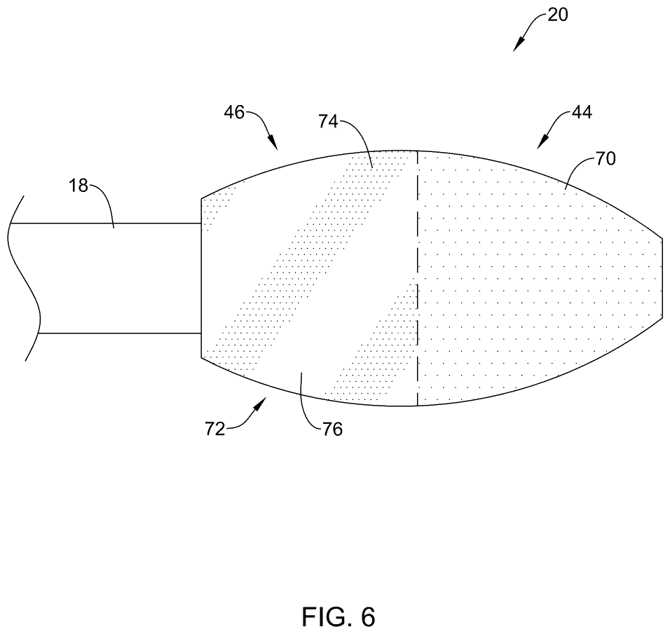

In the examples shown in FIGS. 5A, 5B and 5C, the abrasive particles are laid out in a continuous manner (particular particle size, particle density and overcoat thickness) in the proximal tapered surface 46 and in a continuous manner (possibly different particle size, particle density and overcoat thickness) in the distal tapered surface 44. In some instances, the abrasive particles may be laid out in a continuous manner in the distal tapered surface 44 and may be laid out in a discontinuous manner in the proximal tapered surface 46. FIG. 6 shows the atherectomy burr 20 having a first pattern 70 within the distal tapered surface 44. The first pattern 70 is continuous, with essentially the same average particle size, particle density and overcoat thickness within the first pattern 70. A second pattern 72 is shown within the proximal tapered surface 46. The second pattern 72 is discontinuous. As shown, the second pattern 72 includes stripes 74 that include abrasive particles laid out within an overcoat, as shown in FIGS. 5A, 5B and 5C. The second pattern 72 also includes stripes 76 that are void of abrasive particles. In some instances, the stripes 74 and the stripes 76 may include an overcoat layer that extends through both the stripes 74 and the stripes 76.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An atherectomy burr adapted for use in an atherectomy system, the atherectomy burr comprising:
an atherectomy burr body adapted to be secured to a driveshaft;
the atherectomy burr body having an outer surface defining a distal tapered surface and a proximal tapered surface;
the distal tapered surface having a first abrasiveness adapted for anterograde ablation; and
the proximal tapered surface having a second abrasiveness adapted for retrograde ablation.

2. The atherectomy burr of claim 1, wherein the first abrasiveness is greater than the second abrasiveness.

3. The atherectomy burr of claim 1, wherein the distal tapered surface comprises abrasive particles having a first average particle size and the proximal tapered surface comprises abrasive particles having a second average particle size smaller than the first average particle size.

4. The atherectomy burr of claim 1, wherein the distal tapered surface comprises abrasive particles having a first average exposed particle height and the proximal tapered surface comprises abrasive particles having a second average exposed particle height that is less than the first average exposed particle height.

5. The atherectomy burr of claim 4, wherein the distal surface comprises abrasive particles having an average diameter embedded in a first thickness of overcoat and the proximal surface comprises abrasive particles having the same average diameter embedded in a second thickness of overcoat that is thicker than the first thickness of overcoat.

6. The atherectomy burr of claim 4, wherein the distal surface comprises abrasive particles having a first average diameter embedded in a thickness of overcoat and the proximal surface comprises abrasive particles having a second average diameter embedded in the same thickness of overcoat, where the second average diameter is less than the first average diameter.

7. The atherectomy burr of claim 1, wherein the distal tapered surface comprises abrasive particles having a first particle density value and the proximal tapered surface comprises abrasive particles having a second particle density value that is less than the first particle density value.

8. The atherectomy burr of claim 1, wherein the distal tapered surface comprises abrasive particles laid out in a constant pattern and the proximal tapered surface comprises abrasive particles laid out in a discontinuous pattern.

9. An atherectomy system, comprising:
an advancer assembly;
a drive assembly adapted to translate relative to the advancer assembly;
a knob extending from the drive assembly such that translating the knob results in the drive assembly translating relative to the advancer assembly;
a driveshaft operably coupled with the drive assembly, the driveshaft translating relative to the advancer assembly as the drive assembly translates relative to the advancer assembly; and
the atherectomy burr of claim 1.

10. The atherectomy system of claim 9, wherein the atherectomy burr body defines an axially extending void into which the driveshaft is adapted to be secured.

11. An atherectomy system, comprising:
an advancer assembly;
a drive assembly adapted to translate relative to the advancer assembly;
a knob extending from the drive assembly such that translating the knob results in the drive assembly translating relative to the advancer assembly;
a driveshaft operably coupled with the drive assembly, the driveshaft translating relative to the advancer assembly as the drive assembly translates relative to the advancer assembly; and
an atherectomy burr operably coupled with the driveshaft, the atherectomy burr adapted to limit driveshaft windup during retrograde ablation.

12. The atherectomy system of claim 11, wherein the atherectomy burr comprises a distal tapered surface adapted for anterograde ablation and a proximal tapered surface adapted for retrograde ablation.

13. The atherectomy system of claim 12, wherein the distal tapered surface has a first abrasiveness and the proximal tapered surface has a second abrasiveness that is less than the first abrasiveness.

14. The atherectomy system of claim 13, wherein the distal tapered surface comprises abrasive particles having a first average particle size and the proximal tapered surface comprises abrasive particles having a second average particle size smaller than the first average particle size.

15. The atherectomy system of claim 13, wherein the distal tapered surface comprises abrasive particles having a first average exposed particle height and the proximal tapered surface comprises abrasive particles having a second average exposed particle height that is less than the first average exposed particle height.

16. The atherectomy system of claim 13, wherein the distal tapered surface comprises abrasive particles having a first particle density value and the proximal tapered surface comprises abrasive particles having a second particle density value that is less than the first particle density value.

17. The atherectomy system of claim 13, wherein the distal tapered surface comprises abrasive particles laid out in a constant pattern and the proximal tapered surface comprises abrasive particles laid out in a discontinuous pattern.

18. An atherectomy system, comprising:
an atherectomy burr including a distal outer surface having a first abrasiveness adapted for anterograde ablation and a proximal outer surface having a second abrasiveness adapted for retrograde ablation; and
a drive mechanism adapted to rotatably actuate the atherectomy burr in an anterograde ablation direction and/or in a retrograde ablation direction.

19. The atherectomy system of claim 18, wherein the first abrasiveness is greater than the second abrasiveness.

20. The atherectomy system of claim 18, wherein the drive mechanism comprises:
a drive coil adapted to be coupled with the atherectomy burr; and
a drive assembly adapted to rotatably actuate the drive coil.
